(19) United States
(12) Patent Application Publication
(10) Pub. No.: US 2005/0032757 A1
(43) Pub. Date: Feb. 10, 2005

(54) Nutritional supplements

(76) Inventor: Suk H. Cho, Idaho Falls, ID (US)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/913,848

(22) Filed: Aug. 6, 2004

Related U.S. Application Data
(60) Provisional application No. 60/493,374, filed on Aug. 6, 2003.

(51) Int. Cl. A61K 31/56; A61K 31/202

(52) U.S. Cl. 514/170; 514/560

(57) ABSTRACT

The invention provides compositions containing one or more sterol compounds and one or more fatty acid compounds. The invention also provides methods for reducing CVD risk factors such as LDL cholesterol levels.

### CROSS-REFERENCE TO RELATED APPLICATIONS

This application claims the benefit of and priority under 35 U.S.C. § 119(e) to U.S. Provisional Application 60/493,374, filed Aug. 6, 2003, incorporated herein in its entirety.

### BACKGROUND

1. Technical Field

This invention relates to compositions such as nutritional supplements containing, for example, one or more sterol compounds; to methods for stabilizing mixtures of sterol compounds and fatty acids; and to methods for increasing the bioavailability of sterol compounds or fatty acids.

2. Background Information

Cardiovascular disease (CVD) is a major cause of morbidity and mortality throughout the world. The pathogenesis of CVD is a complex interplay of numerous risk factors, such as a person's lifestyle, diet, and genetic background. While physicians have used various statin drugs, such as lovastatin, pravastatin, and simavastatin to reduce cholesterol levels, the impact of these prescription medications on the heart, liver, and muscles have raised safety concerns. In addition, the use of statins is mainly focused on reducing serum cholesterol levels, while CVD has multiple pathological mechanisms.

### SUMMARY

Typically, the invention provides compositions containing one or more sterol compounds. For example, the invention provides compositions containing a phytosterol. The compositions provided herein can be used as a nutritional supplement. For example, the compositions provided herein can be used to treat or prevent cardiovascular disease or to lower LDL cholesterol levels. In general, the compositions include one or more of the following: a sterol compound (e.g., a phytosterol compound), a fatty acid compound (e.g., an ω-3 fatty acid compound), a carboxylic acid ester (e.g., a small carboxylic acid ester), a surfactant (e.g., lecithin), and an enzyme (e.g., papain) or enzyme blend (e.g., bromelain). Other optional ingredients include antioxidants, reducing agents, and radical scavengers. A composition can be a nutritional supplement. A nutritional supplement can include a label recommending a serving size of at least 1 g of one or more sterol compounds and/or at least 300 mg of one or more fatty acid compounds per day.

The compositions can be in the form of a capsule, e.g., a soft gel or a hard shell capsule. Such capsules can be designed to contain a high percentage of a mixture of one or more sterol compounds with one or more fatty acid compounds. For example, 50, 55, 60, 65, 70, 75, 80, 85, 90, 95, or more percent of the material inside of a capsule can be sterol and fatty acid compounds. A mixture of sterol and fatty acid compounds can be a homogeneous mixture. A ratio of the total sterol compounds to the total fatty acid compounds can be <5:1. In some cases, a capsule can lack beeswax or other typical fillers. In addition, a capsule can provide material that is homogeneous and stable. Further, a capsule described herein can allow the formulated material to be absorbed by the user. For example, a composition can be formulated such that 1, 2, 3, 4, 5, 6, 7, 8, 9, 10, 15, 20, 25, 30, 35, 40, 45, 50, 60, 70, 80, 90, or more percent of a particular compound (e.g., sterol compound or fatty acid compound) formulated within the capsule is absorbed.

In general, a composition can contain one or more sterol compounds (e.g., a phytosterol). A sterol compound can have the following formula: 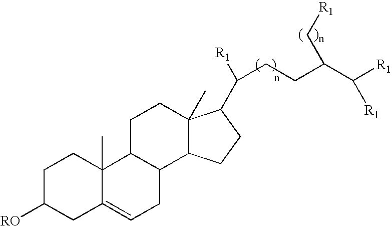
Structure Ia, where R₁ can be —H, a linear or branched, saturated or unsaturated C1, C2, C3, C4, C5, or C6 alkyl, —OH, or —OR″, where R″ can be a linear or branched, saturated or unsaturated C1, C2, C3, C4, C5, or 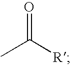
where n can be zero, one, or two; and where R can be H, R′, or 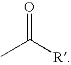
In these embodiments, R′ can be a linear or branched, saturated or unsaturated alkyl chain having 6 to 30 carbon atoms (e.g., 6, 7, 8, 9, 10, 11, 12, 13, 14, 15, 16, 17, 18, 19, 20, 21, 22, 23, 24, 25, 26, 27, 28, 29, or 30 carbon atoms). In certain embodiments, R′ can be a linear or branched, saturated or unsaturated alkyl chain having 12 to 18 carbon atoms (e.g., 12, 13, 14, 15, 16, 17, or 18 carbon atoms).

In other embodiments, a sterol compound can have the following formula: 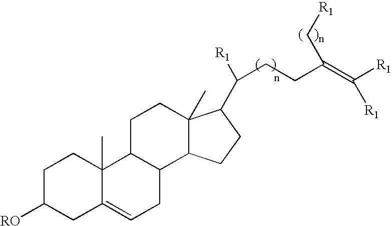
Structure lb, where R1 and R are defined as above.

Examples of compounds according to Structure Ia or Ib include, without limitation, beta- sitosterol laurate ester, alpha-sitosterol laurate ester, gamma-sitosterol laurate ester, campesterol myristearate ester, stigmasterol oleate ester, campesterol stearate ester, beta-sitosterol oleate ester, beta-sitosterol palmitate ester, beta-sitosterol linoleate ester, alpha-sitosterol oleate ester, gamma-sitosterol oleate ester, beta-sitosterol myristerate ester, beta-sitosterol ricinoleate ester, campesterol laurate ester, campesterol ricinoleate ester, campesterol oleate ester, campesterol linoleate ester, stigmasterol linoleate ester, stigmasterol laurate ester, stigmasterol caprate ester, alpha-sitosterol stearate ester, gamma-sitosterol stearate ester, alpha-sitosterol myristearate ester, gamma-sitosterol palmitate ester, campesterol ricinoleate ester, stigmasterol ricinoleate ester, campesterol ricinoleate ester, beta-sitosterol, alpha-sitosterol, gamma-sitosterol, campesterol, stigmasterol, and stigmasterol stearate ester.

In yet other embodiments, a sterol compound can have the following formula: 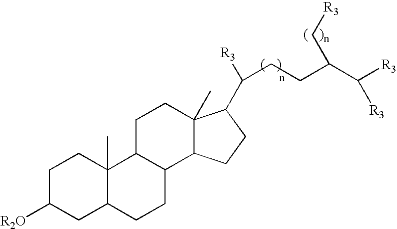
Structure IIa. In these embodiments, R₃ can be —H, a linear or branched, saturated or unsaturated C1, C2, C3, C4, C5, or C6 alkyl, —OH, or —OR“ ”, where R“ ” can be a linear or branched, saturated or unsaturated C1, C2, C3, C4, C5, or C6 alkyl or 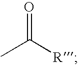
n can be zero, one, or two; and R₂ can be H, R′″ or 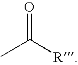
R′″ can be a linear or branched, saturated or unsaturated alkyl chain having 6 to 26 carbon atoms (e.g., 6, 7, 8, 9, 10, 11, 12, 13, 14, 15, 16, 17, 18, 19, 20, 21, 22, 23, 24, 25, or 26 carbon atoms), or, in certain embodiments, 12 to 22 carbon atoms (e.g., 12, 13, 14, 15, 16, 17, 18, 19, 20, 21, or 22).

In some embodiments, a sterol compound can have the following structure: 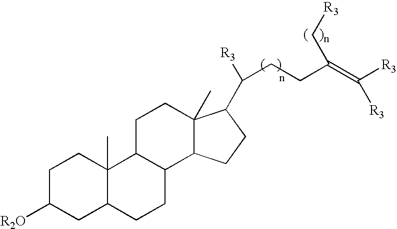
Structure IIb, where R₂ and R₃ are defined as above.

Examples of compounds having Structure IIa or IIb include, without limitation, beta- sitostanol laurate ester, campestanol myristearate ester, stigmastanol oleate ester, campestanol stearate ester, beta-sitostanol oleate ester, beta-sitostanol palmitate ester, beta-sitostanol linoleate ester, beta-sitostanol myristearate ester, beta-sitostanol ricinoleate ester, campestanol laurate ester, campestanol ricinoleate ester, campestanol oleate ester, campestanol linoleate ester, stigmastanol linoleate ester, stigmastanol laurate ester, stigmastanol caprate ester, stigmastanol stearate ester, alpha-sitostanol laurate ester, gamma-sitostanol laurate ester, alpha-sitostanol oleate ester, gamma-sitostanol oleate ester, alpha-sitostanol stearate ester, gamma-sitostanol stearate ester, alpha-sitostanol myristearate ester, gamma-sitostanol palmitate ester, campestanol ricinoleate ester, stigmastanol ricinoleate ester, campestanol ricinoleate ester, beta-sitostanol, alpha-sitostanol, gamma-sitostanol, campestanol, and stigmastanol.

The compositions provided herein can include one or more fatty acid compounds (e.g., one or more ω-3 fatty acid compounds). For example, a composition can contain an ω-3 fatty acid compound having one of the following structures:

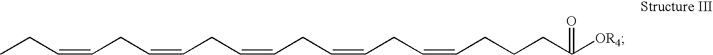

In these embodiments, R4, R₅, and R6 can be, independently, hydrogen; a linear or branched, saturated or unsaturated C2, C3, C4, C5, C6, C7, C8, C9, C10, ClI, or C₁₋₂ alkyl chain; or a 2-hydroxypropyl, hydroxyethyl, tocopheryl, alkyl, or glyceryl (e.g., mono-, di-, or triglyceride) ester. Examples of compounds according to Structures III-V include, without limitation, eicosapentanoic acid (EPA), docosahexanoic acid (DHA), and a-linolenic acid (ALA).

In certain embodiments, a fatty acid compound (e.g., an ω-3 fatty acid compound) can be provided in a fish oil, a vegetable oil, or a mixture thereof. In other embodiments, compounds according to Structures III-V can be in a pure form or can be in a mixture with other compounds. In some cases, the fatty acid compound can be synthesized. A composition provided herein can contain EPA, DHA, and/or ALA, either as components of a fish oil or vegetable oil or in the pure form. In certain embodiments, a ratio of EPA to DHA can range from about 1:5 to about 5:1, or from about 1:2 to about 2:1, or from about 1.5:1 to about 1:1.5.

In some embodiments, a composition described herein can contain a carboxylic acid ester (e.g., a small carboxylic acid ester) having the following structure: 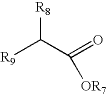
Structure VI, where R₇ can be a linear or branched, saturated or unsaturated alkyl group having 1 to 12 carbon atoms (e.g., methyl, ethyl, butyl, or isopropyl); R₈ and R9 can be, independently, —H; —OH; —OR, wherein R is a linear or branched alkyl group having 1 to 10 carbon atoms (e.g., 1, 2, 3, 4, 5, 6, 7, 8, 9, or 10 carbon atoms); an acyl group (e.g., acetate, propionate, or butyrate); or a linear or branched C1, C2, C3, C4, C5 or C6 alkyl group containing a carboxylate ester. Examples of carboxylic acid esters include, without limitation, ethyl lactate, propyl acetate, triethyl citrate, diethyl maleate, diethylmalonate, diethyl succinate, triisopropyl citrate, diethyl tartrate, propyl lactate, butyl acetate, tripropylcitrate, diisopropyl maleate, dibutylmalonate, dipropyl succinate, trimethyl citrate, and dihexyl tartrate.

Compositions provided herein can contain one or more enzymes. For example, a composition can contain bromelain, papain, fungal proteases, acid stable proteases, neutral stable proteases, alkaline stable proteases, or mixtures thereof.

In certain embodiments, a composition provided herein can contain a surfactant, such as a food grade surfactant. A surfactant can have a surface tension below 70 dyne/cm², or below 40 dyne/cm². A surfactant can have a hydrophilic/lipophilic balance of less than 20, or less than 10. Examples of surfactants include, without limitation, alkanoylglycerides, monoacylglycerides, or monoglycerides (e.g., from rapeseed, canola, and cottonseed oils); propylene glycol monoesters (e.g., propylene glycol monostearate); lactoylesters; stearic acid; sodium stearoyl lactylate; ethoxylated alcohols; ethoxylated fatty esters and fatty esters; ethyoxylated glycerol esters; phosphorous organic derivatives such as dodecyl phosphonic acid, dodecyl phosphate, decylphosphonic acid, decyl phosphate, dioctylphosphate, myristearylphosphonic acid, lecithin and lecithin derivatives; sorbitan derivatives such as polyoxyethylene sorbitan monolaurate, sorbitan oleate, sorbitan laurate, sorbitan palmitate, sorbitan stearate, sorbitan myristearate, sorbitan ricinoleate, sorbitan linoleneate, and sorbitan linoleate; stearoyl-2-lactylates of sodium or calcium; sucrose and glucose esters and derivatives thereof; sulfosuccinates and derivatives; and mixtures of any of the above. Sorbitan derivatives and phosphorous organic derivatives, such as lecithin, can be used to increase stability of a composition or to increase bioavailability of a sterol compound (e.g., a plant phytosterol) and/or a fatty acid compound (e.g., a α-3 fatty acid compound). The surfactant can be included at a concentration of about 0.01% to about 15%, or from about 0.1% to about 10%, or from about 0.1% to about 7% by weight of the composition.

In other embodiments, the compositions provided herein can contain an agent such as a radical scavenger, an antioxidant, a reducing agent, and/or mixtures thereof. For example, the agent can be an antidoxidant such as ascorbic acid, tocopheryl acetate, CoQ-10, tocopheryl palmitate, butyl hydroxytoluene, tocotrienol, retinyl palmitate, betacarotene, xeaxanthine, lutene, lycopene, retinyl acetate, or polyphenolic-containing herbs and plant matter (such as green tea extracts or grape seed extracts). A reducing agent can be sodium bisulfite.

The compositions provided herein can be formulated as nutritional supplements, e.g., in the form of liquids, pastes, and capsules. A nutritional supplement can contain a flavoring and/or coloring agent.

In one aspect, the invention provides a capsule containing a phytosterol compound and an ω-3 fatty acid compound. The capsule can contain a small carboxylic acid ester, a surfactant, an enzyme or enzyme blend, and/or an agent selected from the group consisting of a radical scavenger, an antioxidant, a reducing agent, and mixtures thereof. The phytosterol compound can be selected from the group consisting of beta-sitosterol laurate ester, alpha-sitosterol laurate ester, gamma-sitosterol laurate ester, campesterol myristearate ester, stigmasterol oleate ester, campesterol stearate ester, beta-sitosterol oleate ester, beta-sitosterol palmitate ester, beta- sitosterol linoleate ester, alpha-sitosterol oleate ester, gamma-sitosterol oleate ester, beta- sitosterol myristearate ester, beta-sitosterol ricinoleate ester, campesterol laurate ester, campesterol ricinoleate ester, campesterol oleate ester, campesterol linoleate ester, stigmasterol linoleate ester, stigmasterol laurate ester, stigmasterol caprate ester, alpha-sitosterol stearate ester, gamma-sitosterol stearate ester, alpha-sitosterol myristearate ester, gamma-sitosterol palmitate ester, campesterol ricinoleate ester, stigmasterol ricinoleate ester, campesterol ricinoleate ester, beta-sitosterol, alpha-sitosterol, gamma-sitosterol, campesterol, stigmasterol, stigmasterol stearate ester, beta-sitostanol laurate ester, campestanol myristearate ester, stigmastanol oleate ester, campestanol stearate ester, beta-sitostanol oleate ester, beta-sitostanol palmitate ester, beta- sitostanol linoleate ester, beta-sitostanol myristearate ester, beta-sitostanol ricinoleate ester, campestanol laurate ester, campestanol ricinoleate ester, campestanol oleate ester, campestanol linoleate ester, stigmastanol linoleate ester, stigmastanol laurate ester, stigmastanol caprate ester, stigmastanol stearate ester, alpha-sitostanol laurate ester, ganima-sitostanol laurate ester, alpha- sitostanol oleate ester, gamma-sitostanol oleate ester, alpha-sitostanol stearate ester, gamma- sitostanol stearate ester, alpha-sitostanol myristearate ester, gamma-sitostanol palmitate ester, campestanol ricinoleate ester, stigmastanol ricinoleate ester, campestanol ricinoleate ester, beta- sitostanol, alpha-sitostanol, gamma-sitostanol, campestanol, and stigmastanol. The ω-3 fatty acid compound can be selected from the group consisting of EPA, DHA, and ALA. The ω-3 fatty acid compound can be provided in the form of a fish oil or a vegetable oil. The vegetable oil can be selected from the group consisting of canola oil, rapeseed oil, cottonseed oil, and flaxseed oil. The phytosterol compound can be a phytosterol ester. The small carboxylic acid ester can be selected from the group consisting of ethyl lactate, propyl acetate, triethyl citrate, diethyl maleate, diethylmalonate, diethyl succinate, triisopropyl citrate, diethyl tartrate, propyl lactate, butyl acetate, tripropylcitrate, diisopropyl maleate, dibutylmalonate, dipropyl succinate, trimethyl citrate, and dihexyl tartrate. The surfactant can be selected from the group consisting of lecithin, lecithin derivatives, polyoxyethylene sorbitan monolaurate, sorbitan oleate, sorbitan laurate, sorbitan palmitate, sorbitan stearate, sorbitan myristearate, sorbitan ricinoleate, sorbitan linoleneate, and sorbitan linoleate.

In another embodiment, the invention features a capsule containing a phytosterol compound and a fish oil. The capsule can contain a small carboxylic acid ester.

In another embodiment, the invention features a capsule containing a phytosterol compound and a vegetable oil. A capsule can contain a small carboxylic acid ester.

Another embodiment of the invention features a capsule containing a phytosterol compound and a mixture containing a vegetable oil and a fish oil. The capsule can contain a small carboxylic acid ester.

Another embodiment of the invention features a capsule containing (a) about 45% to about 95% by weight of a phytosterol compound; (b) about 10% to about 55% by weight of an α-3 fatty acid compound; and (c) about 0.01% to about 15% by weight of a small carboxylic acid ester. The capsule can contain about 0.01% to about 15% by weight of a surfactant. The small carboxylic acid ester can be triethyl citrate. The surfactant can be a sorbitan derivative.

Another embodiment of the invention features a method for reducing a detectable measure of a CVD risk factor in a mammal. The method includes administering capsules described herein to the mammal, wherein the capsules are administered in an amount, at a frequency, and for a duration effective to reduce the detectable measure of the CVD risk factor. The CVD risk factor can be a LDL cholesterol level.

Another embodiment of the invention features a method for increasing bioavailability of a phytosterol compound. The method includes incorporating a small carboxylic acid ester in a composition containing the phytosterol compound and an ω-3 fatty acid compound. The method can include incorporating a surfactant in the composition.

Another embodiment of the invention features a method for stabilizing a mixture of a phytosterol compound and an ω-3 fatty acid compound. The method includes incorporating a small chain carboxylic acid ester in the mixture.

Another embodiment of the invention features a soft gel capsule lacking beeswax, wherein the soft gel capsule containing a mixture of a phytosterol compound and an ω-3 fatty acid compound. The soft gel capsule can contain a small chain carboxylic acid ester. The soft gel capsule can contain a surfactant.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the description below. Other features, objects, and advantages of the invention will be apparent from the drawings and detailed description, and from the claims.

### DETAILED DESCRIPTION

The invention provides compositions that can contain (1) one or more sterol compounds; (2) one or more fatty acid compounds; or (3) a mixture of one or more sterol compounds and one or more fatty acid compounds. For example, a composition provided herein can contain a phytosterol compound and an ω-3 fatty acid compound. A composition can have a ratio of the total sterol compounds to total fatty acid compounds of <5:1 (e.g., about 4.5:1, 4:1, 3.5:1, 3:1, 2.5:1, 2:1, 1.5:1, 1:1, or 0.5:1). A composition can be provided as a nutritional supplement with a label recommending a serving size of at least 1 g (e.g., 1 g, 1.3 g, 1.5 g, 1.7 g, 1.8 g, 2.0 g, 2.5 g, 2.75 g, 3.0 g, 3.25 g, or 3.5 g) of the one or more sterol compounds and/or at least 300 mg of the one or more fatty acid compounds (e.g., ω-3 fatty acid compounds) per day. The compositions can contain other ingredients such as one or more small chain carboxylic acid esters, one or more surfactants, one or more enzymes or enzyme blends, one or more antioxidants, one or more reducing agents, or one or more radical scavengers. For example, a composition provided herein can contain a phytosterol compound and a small carboxylic acid ester. Certain compositions can contain an ω-3 fatty acid compound and a small carboxylic acid ester. Some compositions can contain a phytosterol compound, an ω-3 fatty acid compound, and a small carboxylic acid ester. Certain compositions can contain a phytosterol compound, an ω-3 fatty acid compound, and an enzyme or enzyme blend. Compositions having a phytosterol compound and a ω-3 fatty acid compound can contain an antioxidant. A surfactant can be included in any of the compositions described herein. For example, a composition can contain a phytosterol compound, an ω-3 fatty acid compound, a small carboxylic acid ester, an antioxidant, a surfactant, and an enzyme or enzyme blend. Typically, the compositions are formulated as soft gel capsules or hard shell capsules without the addition of beeswax or other fillers. Soft gel cap or hard shell caplet formulations described herein can exhibit improved stability and homogeneity of the material contained within the soft gel cap or hard shell caplet.

Sterol Compounds

Compositions provided herein can contain a sterol compound such as a phytosterol compound. In some cases, the sterol can be synthetic. The term “sterol” includes, without limitation, plant, animal, and synthetic sterols, sterol esters, stanols, and stanol esters. Plant sterols (and sterol esters) are naturally occurring substances present in the diet as minor components of vegetable oils, while plant stanols (and stanol esters) are hydrogenation compounds of the plant sterols.

A sterol compound can have the following structure: 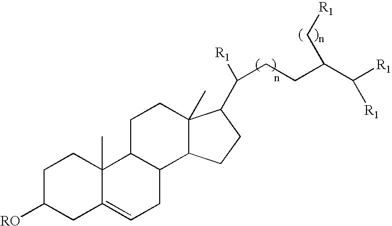
Structure Ia, where R₁ can be —H, a linear or branched, saturated or unsaturated C1, C2, C3, C4, C5, or C6 alkyl, —OH, or —OR″, where R″ can be a linear or branched, saturated or unsaturated C1, C2, C3, C4, C5, or C6 alkyl or 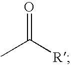
where n can be zero, one, or two; and where R can be H, R′, or

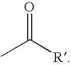

Alternatively, a sterol compound can have the following structure: 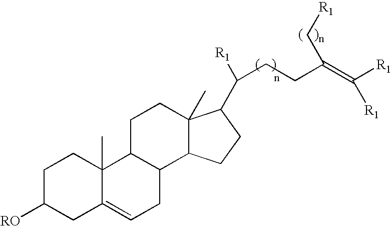
Structure Ib, where R₁ and R are defined as in Structure Ia above.

Some compounds according to Structures Ia or Ib can be called phytosterols or phytosterol esters. In addition, they can be provided in a free alcohol (sterol) or esterified form. When R is H, the compound can be referred to as a sterol. On the other hand, when R in Structure Ia or Ib is 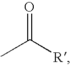
the compound can be called a sterol ester. In these embodiments, R′ can be a linear or branched, saturated or unsaturated alkyl chain having 6 to 30 carbon atoms. In certain embodiments, R′ is a linear or branched, saturated or unsaturated alkyl chain having 12 to 18 carbon atoms.

Sterol compounds (e.g., phytosterols and phytosterol esters) can be obtained commercially from, e.g., Cargill, Inc. (Minneapolis, Minn.), Loders and Croklaan (Channahon, Ill.), Cognis Nutrition and Health (La Grange, Ill.), Forbes Meditech (Vancouver, B.C. Canada), and ADM (Decatur, Ill.) and can demonstrate a range of sterol profiles. For example, Vegapure™ 95 sterol esters from Cognis Nutrition and Health is a mixture of sterol esters produced from a mixture of soy, rapeseed and other vegetable oil distillates. Vegapure™ 95 can include at least 90% (e.g. about 91%, 92%, 93%, 94%, 95%, 96%, 97%, 98%, or 99%) free sterols and sterol esters, with β-sitosterol ester ranging from about 40% to about 60% by weight, campesterol ester ranging from about 20% to about 40% by weight, stigmasterol ester ranging from about 12% to about 23% by weight, brassicasterol ester ranging from about 0 to about 12% by weight, and free sterols ranging from 0% to about 6% by weight. CoroWise™ Phytosterol Esters from Cargill, Inc. has a typical phytosterol content of greater than 88%, with sitosterol ranging from about 40-58%, campesterol ranging from about 20-28%, and stigmasterol ranging from about 14-23%. ADM Phytosterols 040095 include a mixture of sterols having about 40-58% by weight β-sitosterol, 20-30% by weight campesterol, 14-22% by weight stigmasterol, 0-6% by weight brassicasterol, 0-5% by weight sitostanol, and total phytosterols at a minimum of 90% by weight. In addition, sterol compounds can be synthesized and/or obtained from natural sources such as soy oil, canola oil, or wheat germ oil as described elsewhere (U.S. Pat. Nos. 6,411,206; 5,502,045; 6,087,353; and 4,897,224).

Suitable examples of phytosterol esters that can be used to prepare a composition provided herein include, without limitation, beta-sitosterol laurate ester, alpha-sitosterol laurate ester, gamma-sitosterol laurate ester, campesterol myristearate ester, stigmasterol oleate ester, campesterol stearate ester, beta-sitosterol oleate ester, beta-sitosterol palmitate ester, beta-sitosterol linoleate ester, alpha-sitosterol oleate ester, gamma-sitosterol oleate ester, beta-sitosterol myristearate ester, beta-sitosterol ricinoleate ester, campesterol laurate ester, campesterol ricinoleate ester, campesterol oleate ester, campesterol linoleate ester, stigmasterol linoleate ester, stigmasterol laurate ester, stigmasterol caprate ester, alpha-sitosterol stearate ester, gamma-sitosterol stearate ester, alpha-sitosterol myristearate ester, gamma-sitosterol palmitate ester, campesterol ricinoleate ester, stigmasterol ricinoleate ester, campesterol ricinoleate ester, beta-sitosterol, alpha-sitosterol, gamma-sitosterol, campesterol, stigmasterol, and stigmasterol stearate ester.

In some embodiments, a sterol compound can have the following structure: 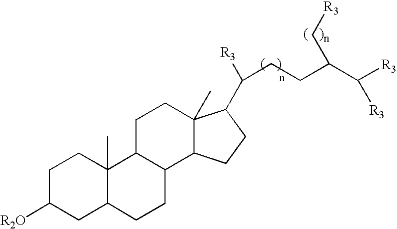
Structure IIa. In these embodiments, R₃ can be-H, a linear or branched, saturated or unsaturated C1, C2, C3, C4, C5, or C6 alkyl, —OH, or —OR″″, where R″″ can be a linear or branched, saturated or unsaturated C1, C2, C3, C4, C5, or C6 alkyl or 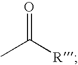
n can be zero, one, or two; and R₂ can be H, R′″ or

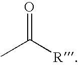

In some cases, a sterol compound can have the following formula:

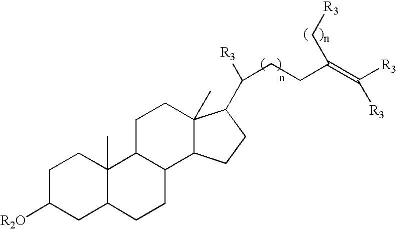

Structure IIb, where R₂ and R₃ are defined as for Structure IIa above.

When R₂ is H, the compound can be referred to as a phytostanol. When R₂ is 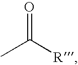
the compound can be called a phytostanol ester. In these embodiments, R′″ can be a linear or branched, saturated or unsaturated alkyl chain having 6 to 26 carbon atoms. In certain embodiments, R′″ can be a linear or branched, saturated or unsaturated alkyl chain having 12 to 22 carbon atoms.

Sterol compounds such as phytostanols and phytostanol esters can be obtained commercially from, e.g., Forbes Meditech (Vancouver, B.C. Canada), or can be readily synthesized. Alternatively, phytostanols and phytostanol esters can be obtained from natural sources such as soy oil, canola oil, or wheat germ oil as described elsewhere (U.S. Pat. Nos. 6,411,206; 5,502,045; 6,087,353; and 4,897,224).

Examples of phytostanol esters that can be used to make a composition provided herein include, without limitation, beta-sitostanol laurate ester, campestanol myristearate ester, stigmastanol oleate ester, campestanol stearate ester, beta-sitostanol oleate ester, beta-sitostanol palmitate ester, beta-sitostanol linoleate ester, beta-sitostanol myristearate ester, beta-sitostanol ricinoleate ester, campestanol laurate ester, campestanol ricinoleate ester, campestanol oleate ester, campestanol linoleate ester, stigmastanol linoleate ester, stigmastanol laurate ester, stigmastanol caprate ester, stigmastanol stearate ester, alpha-sitostanol laurate ester, gamma-sitostanol laurate ester, alpha-sitostanol oleate ester, gamma-sitostanol oleate ester, alpha-sitostanol stearate ester, gamma-sitostanol stearate ester, alpha-sitostanol myristearate ester, gamma-sitostanol palmitate ester, campestanol ricinoleate ester, stigmastanol ricinoleate ester, campestanol ricinoleate ester, beta-sitostanol, alpha-sitostanol, gamma-sitostanol, campestanol, and stigmastanol.

Sterol compounds of the compositions provided herein can have a melting point below 145° C. In other embodiments, the sterol compounds can have a melting point below 85° C., below 65° C., or below 45° C.

A composition can contain any type of sterol compound such as a phytosterol, phytosterol ester, phytostanol, or phytostanol ester. In addition, a composition can contain any amount of sterol compounds (e.g., 10, 25, 50, 100, 200, 250, 300, 325, 350, 375, 400, 425, 450, 475, 500, 525, 550, 575, 600, 625, 650, 675, 700, 750, 800, or 900 mg). For example, between 35 to 95 percent (e.g., from 35 to 95 percent, from 50 to 95 percent, from 75 to 95 percent, from 85 to 95 percent, from 35 to 85 percent, from 35 to 75 percent, or from 35 to 55 percent) of a composition can contain sterol compounds. In some cases, a composition can be designed to contain multiple sterol compounds in any relative ratio (e.g., 1:1, 2:1, 3:1, 4:1, 5:1, 6:1, 7:1, 8:1, 9:1, or 10:1). In certain cases, a composition can be a nutritional supplement with a label indicating that a suggested serving size includes greater than 1 g (e.g., about 1.1, 1.2, 1.3, 1.4, 1.5, 1.6, 1.7, 1.8, 1.9, 2.0, 2.1, 2.2, 2.3, 2.4, 2.5, 2.6, 3.0, 3.5 g or more) of total sterol compounds per day.

Typically, a composition can be designed to contain a phytosterol ester compound or mixtures thereof. In addition, a composition can be designed to contain one or more phytosterol ester compounds in a total amount ranging from about 45% to about 95% by weight of the composition. In certain embodiments, one or more phytosterol ester compounds can be provided in a total amount from about 45% to about 60% by weight of the composition. In other embodiments, one or more phytosterol ester compounds can be provided in a total amount of about 70% to about 85% by weight of the composition.

A composition provided herein can contain one or more phytosterol compounds (e.g., a compound according to Structure Ia or Ib where R in Structure I is H). In certain embodiments, one or more phytosterols can be present in a total amount from about 1% to about 85% by weight of the composition. In certain compositions, one or more phytosterol compounds may be present in a total amount from about 1% to about 10% by weight of the composition. In other compositions, one or more phytosterols can be present in a total amount of from about 40% to about 55%, or from about 70% to about 85% by weight of the composition.

In certain embodiments, a composition provided herein can contain one or more phytostanol ester compounds, e.g., in a total amount of from about 45% to about 95% by weight of the composition. In certain embodiments, one or more phytostanol ester compounds can be provided in a total amount of about 45% to about 60% by weight of the composition. In other embodiments, one or more phytostanol ester compounds can be provided in a total amount of about 70% to about 85% by weight of the composition.

In certain compositions, one or more phytostanol compounds can be present in a total amount from about 1% to about 85% by weight of the composition. In other compositions, one or more phytostanols can be present in a total amount of from about 1% to about 10%, from about 40% to about 55%, or from about 70% to about 85% by weight of the composition.

Fatty Acid Compounds

The compositions provided herein can contain one or more fatty acid compounds (e.g., ω-3 fatty acid compounds). An ω-3 fatty acid for use in the present invention can have the following structure: 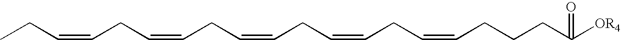
Structure III. ω-3 fatty acids can generally be in the simple form (e.g., where R₄ is H) or alternatively in the glyceride ester form. For example, R₄ can be hydrogen; a linear or branched, saturated or unsaturated C₂-C₁₂ alkyl chain; or a 2-hydroxypropyl, hydroxyethyl, tocopheryl, alkyl, or glyceryl (e.g., mono-, di-, or triglyceride) ester. Compounds according to Structure III are generally known as eicosapentanoic acid (EPA) and derivatives thereof.

In some embodiments, an ω-3 fatty acid compound can have the following structure: 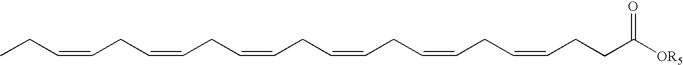
Structure IV, where R₅ can be hydrogen; a linear or branched, saturated or unsaturated C₂-C₁₂ alkyl chain; or a 2-hydroxypropyl, hydroxyethyl, tocopheryl, alkyl, or glyceryl (e.g., mono-, di-, or triglyceride) ester. Compounds according to Structure IV are known as docosahexanoic acid (DHA) and derivatives thereof.

In other embodiments, an ω-3 fatty acid compound can have the following structure: 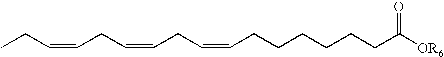
Structure V, where R₆ can be hydrogen; a linear or branched, saturated or unsaturated C2-C12 alkyl chain; or a 2-hydroxypropyl, hydroxyethyl, tocopheryl, alkyl, or glyceryl (e.g., mono-, di-, or triglyceride) ester. Compounds according to Structure V are known as α-linolenic acid (ALA) and derivatives thereof.

Fatty acid compounds such as α-3 fatty acid compounds can be obtained from fish oils or vegetable oils, or synthesized. In some cases, fatty acid compounds such as ω-3 fatty acid compounds can be formulated in a composition as a fish oil or vegetable oil or mixture thereof. For example, a fish oil containing DHA, EPA, or both, and/or a vegetable oil containing ALA (or derivatives thereof) can be used to make a composition provided herein. Fish oils are available commercially from BLT Berg Lipidtech AS, Clover Corporation, Denofa AS, Bioriginal Food and Science Corp., ProNova Biocare, BASF, and NutriScience Innovations LLC, and can demonstrate a range of ω-3 fatty acid profiles. In certain cases, a fish oil can contain about 11-14% DHA and about 16-19% EPA, and a total ω-3 fatty acid content of about 33-41%. For example, a fish oil can contain about 12% DHA and about 18% EPA. In other cases, a fish oil can contain about 30-33% EPA and about 20-22% DHA, and a total α-3 fatty acid content of about 50-67%. In other cases, a fish oil can contain about 50-55% DHA and about 5-10% EPA. In yet other cases, a fish oil can contain about 50-55% EPA and about 5-10% DHA. In other examples, a fish oil can contain at least 20% DHA and at least 25% EPA, and a total ω-3 fatty acid content of at least 60%, e.g., at least 65%. In yet other cases, a fish oil can contain about 5% EPA and about 25% DHA, and a total ω-3 fatty acid content of about 30%, e.g., about 35%. A vegetable oil can contain greater than 10% ω-3 fatty acids by weight, or greater than 25% ω-3 fatty acids by weight. Any type of vegetable oil can be used including, without limitation, canola, flaxseed, or rapeseed oil.

In addition to use of a pure fish oil or vegetable oil, a fish oil or vegetable oil containing EPA, DHA, and/or ALA (or derivatives thereof) can be modified by, e.g., the addition of purified EPA, DHA, or ALA (or derivatives thereof) to result in a particular ratio or amount of EPA, DHA, or ALA (or derivatives thereof). In other embodiments, purified DHA, EPA, or ALA, or derivatives thereof, can be used to make a composition provided herein. In certain embodiments, a ratio of EPA to DHA, or derivatives thereof, can be from about 1:5 to about 5:1, or from about 1:2 to about 2:1, or from about 1.5:1 to about 1:1.5. For example, a fish oil can contain EPA and DHA at a ratio of about 1.5:1.

Typically, a composition provided herein can contain a fish oil or a vegetable oil, or both, in a range from about 10% to about 55% by weight of the composition, or from about 10% to about 30% by weight of the composition. In other embodiments, EPA, DHA, and/or ALA, or derivatives thereof, can be used in purified form to make a composition provided herein. The combined amount of EPA and DHA (or derivatives thereof) can range from about 30% to about 55% by weight of the composition. In certain embodiments, the combined amount of EPA and DHA can range from about 20% to about 40% by weight of the composition. In other embodiments, the combined amount of EPA and DHA can range from about 12% to about 16% by weight of the composition.

Fish oils, vegetable oils, purified EPA, DHA, and ALA, and derivatives thereof such as DHA, EPA, or ALA esters or glycerides can be obtained commercially from, for example, Croda, Inc. (Parsippany, N.J.), Roche Vitamins Ltd. (Parsippany, N.J.), Martek (Boulder, Colo.), Maritex (Sortland, Norway), Seven Seas, Pronova (Lysker, Norway), and Loder Croklann Lipid Nutrition (Channahon, Ill.), and Cargill, Inc. (Minneapolis, Minn.). In certain embodiments, ω-3 fatty acids, particularly those according to Structure V (e.g., ALA) can be provided in a flax seed oil mixture, a canola oil mixture, a walnut oil mixture, or mixtures thereof.

A composition can be a nutritional supplement. A nutritional supplement can include a label indicating that a suggested serving size includes at least 300 mg (e.g., about 300, 350, 400, 420, 450, 500, 500-600, 600-900, 1000, 1100, 1200, 900-1200, 1200-1500 mg, 1500 mg-2 g, 2 g-2.5 g, 2.5 g-3 g, or more) of total ω-3 fatty acids per day. One or more ω-3 fatty acids can be derived from one or more fish or vegetable oils, purified ω-3 fatty acids, or mixtures thereof. In some embodiments, a nutritional supplement can include a label indicating that a suggested serving size includes at least 300 mg of DHA and EPA per day.

In embodiments containing one or more phystosterol compounds and one or more α-3 fatty acid compounds, a ratio of the combined amount of sterol compounds (e.g., phytosterol compounds) relative to the combined amount of fatty acid compounds (e.g., ω-3 fatty acid compounds) can be ≦about 5:1 (e.g., about 4:1, 3:1, 2:1, or 1.5:1).

Carboxylic Acid Esters

Compositions described herein can contain one or more carboxylic acid esters (e.g., a small carboxylic acid ester) having the following structure: 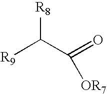
Structure VI, where R₇ can be a linear or branched, saturated or unsaturated alkyl group having 1 to 12 carbon atoms (e.g., methyl, ethyl, butyl, isopropyl); R₈ and R₉ can be, independently, —H, —OH; —OR, wherein R is a linear or branched alkyl group having 1 to 10 carbon atoms; an acyl group (e.g., acetate, propionate, butyrate); or a linear or branched C₁-C₆ alkyl group containing a carboxylate ester.

Examples of small carboxylic acid esters include, without limitation, ethyl lactate, propyl acetate, triethyl citrate, diethyl maleate, diethylmalonate, diethyl succinate, triisopropyl citrate, diethyl tartrate, propyl lactate, butyl acetate, tripropylcitrate, diisopropyl maleate, dibutylmalonate, dipropyl succinate, trimethyl citrate, and dihexyl tartrate.

Typically, small carboxylic acid esters can be present in an amount from about 0.01% to about 15% by weight of a composition, from about 0.1 to about 10%, or from about 0.5 to about 5% by weight of the composition.

Enzymes and Enzyme Blends

Compositions provided herein can contain an enzyme or an enzyme blend. Examples of enzymes or enzyme blends include, without limitation, bromelain, papain, lungal proteases, acid stable proteases, neutral stable proteases, alkaline stable proteases, and mixtures thereof. The enzyme or enzyme blend can be derived from animals, plants, or fungi. Generally, the enzyme or enzyme blend is provided in an amount ranging from about 0.1% to about 5% by weight of the composition, or from about 0.5% to about 2.5% by weight of the composition.

In certain embodiments, the enzyme blend bromelain is used. Bromelain is the generic name for a family of sulfhydryl-containing proteolytic enzymes obtained from the pineapple plant. A bromelain enzyme blend generally contains a sulfydryl proteolytic fraction, a peroxidase, an acid phosphatase, several protease inhibitors, and calcium.

Enzymes or enzyme blends can be obtained commercially from, for example, National Enzyme Company (Forsyth, Mo.), American Laboratories, Inc. (Omaha, Nebr.), Botanical International (Long Beach, Calif.) or Marcor Development Corporation (Carlstadt, N.J.). Enzymes or enzyme blends used to make a composition provided herein can be food and/or pharmaceutical grade.

Surfactants

The compositions provided herein can be designed to contain a surfactant that wets, solubilizes, and/or emulsifies lipophilic components such as sterol compounds (e.g., phytosterol compounds) and/or fatty acid compounds (e.g., fish oil components and ω-3 fatty acids such as EPA, DHA, or ALA). Typically, a surfactant is a food grade surfactant. A surfactant can be anionic, cationic, zwitterionic, or non-ionic. In certain embodiments, a surfactant can have a surface tension below 70 dyne/cm², or below 40 dyne/cm². A surfactant can have a hydrophilic/lipophilic balance of less than 20, or less than 10. See, e.g., Surfactants in Chemistry, J. Falbe, Ed., Springer-Verlag (1989), pp: 149-152.

One or more surfactants can be used in any combination or relative ratio. Examples of surfactants include, without limitation, alkanoylglycerides, monoacylglycerides, or monoglycerides (e.g., from rapeseed, canola, and cottonseed oils); propylene glycol monoesters (e.g., propylene glycol monostearate); lactoylesters; stearic acid; sodium stearoyl lactylate; ethoxylated alcohols; ethoxylated fatty esters and fatty esters; ethyoxylated glycerol esters; phosphorous organic derivatives such as dodecyl phosphonic acid, dodecyl phosphate, decylphosphonic acid, decyl phosphate, dioctylphosphate, myristearylphosphonic acid, lecithin and lecithin derivatives; sorbitan derivatives such as polyoxyethylene sorbitan monolaurate, sorbitan oleate, sorbitan laurate, sorbitan palmitate, sorbitan stearate, sorbitan myristearate, sorbitan ricinoleate, sorbitan linoleneate, and sorbitan linoleate; stearoyl-2-lactylates of sodium or calcium; sucrose and glucose esters and derivatives thereof; sulfosuccinates and derivatives; and mixtures of any of the above. Sorbitan derivatives and phosphorous organic derivatives, such as lecithin, can be used as surfactants to increase stability of a composition, particularly a composition formulated as a soft gel cap or hard shell caplet, or to increase bioavailability of a sterol compound (e.g., a phytosterol compound) and/or a fatty acid compound (e.g., a ω-3 fatty acid compound). A surfactant can be included at a concentration of about 0.01% to about 15%, or from about 0.1% to about 10%, or from about 0.1% to about 7% by weight of the composition.

Surfactants can be obtained commercially as described elsewhere (“McCutcheon's Emulsifiers and Detergents,” Int'l Ed. (2001), The Manufacturing Confectioner Publishing Co., NJ, USA). Typically, a surfactant is present in an amount from about 0.01% to about 15% by weight of a composition, or from about 0.1 to about 10%, or from about 0.1 to about 7% by weight of the composition.

Radical Scavengers, Antioxidants, and Reducing Agents

The compositions provided herein can contain an agent such as a radical scavenger, an antioxidant, a reducing agent, and mixtures thereof. Radical scavengers, antioxidants, and reducing agents can be obtained commercially from common suppliers. Sodium bisulfate can be used as a reducing agent. Examples of radical scavengers and antioxidants include, without limitation, ascorbic acid, tocopheryl acetate, Coenzyme Q-10, tocopheryl palmitate, tocotrienol, retinyl palmitate, betacarotene, zeaxanthine, lutene, lycopene, retinyl acetate, polyphenolic-containing herbs or plant matter (e.g., green tea extracts or grape seed extracts), and butyl hydroxytoluene. Coenzyme Q-10 (CoQ-10) is a fat-soluble quinone that is structurally similar to vitamin K and is commonly known as ubiquinone. Mixtures of one or more radical scavengers, antioxidants, and reducing agents can be used. A radical scavenger, antioxidant, and/or reducing agent can be included in an amount ranging from about 0.01% to about 2% by weight of the composition, or from about 0.1% to about 1% by weight.

Formulations and Optional Ingredients

Compositions described herein can contain additional optional ingredients. For example, optional coloring and/or flavoring agents, e.g., to reduce the odor associated with fish oil and fish oil components, can be included. In addition, the compositions can contain a pharmaceutically acceptable carrier for in vivo administration to a mammal, including, without limitation, preservatives and other additives such as, for example, botanical extracts.

Compositions provided herein can be formulated as nutritional supplements, e.g., as liquids or capsules for oral ingestion. Capsules can be hard-shell or soft gel capsules. The nutritional supplements provided herein can include an effective amount of, for example, sterol compounds (e.g., phytosterol compounds) and/or fatty acid compound (e.g., ω-3 fatty acid compounds), without the addition of non-nutritive ingredients, such as protein complexes or fillers such as beeswax. In addition, the nutritional supplements can exhibit improved formulation stability (e.g., do not separate) and can demonstrate improved bioavailability of sterol compounds (e.g., phytosterol compounds) and/or fatty acid compound (e.g., ω-3 fatty acid compounds) without the need for inclusion of undesirable fillers (e.g., beeswax) or saturated or trans fats.

Methods for Treating or Preventing CVD and Reducing CVD Risk Factors

The compositions provided can be used to reduce inflammatory responses associated with CVD and can be used to reduce CVD risk factors such as plasma total cholesterol levels, LDL cholesterol levels, and triglyceride levels. For example, the compositions provided herein can be used to reduce the levels of C-reactive protein, to reduce or inhibit platelet aggregation, and to reduce or inhibit LDL cholesterol oxidation. In addition, the components of the compositions can be used in ratios and relative amounts that synergistically reduce CVD risk factors such as plasma cholesterol, LDL cholesterol, or triglyceride levels. The compositions can be used alone or in conjunction with prescription drugs, e.g., statin or fibrate drugs.

Typically, a composition is provided to a mammal, e.g., a human, in an amount and for a sufficient time period effective to reduce a detectable measure of a CVD risk factor. A detectable measure of a CVD risk factor can be a mammal's serum or plasma level of C-reactive protein, total plasma cholesterol level, LDL cholesterol level, or triglycerides level. In addition, a composition can be provided to a mammal in an amount and for a sufficient time period effective to reduce or inhibit the mammal's platelet activity level in manner detectable using a platelet aggregation assay or a mammal's LDL cholesterol oxidation rate in a manner detectable using an LDL oxidation assay. Platelet aggregation and LDL oxidation assays are described elsewhere (Flavonoids and Other Polyphenols, Methods of Enzymology, Volume 335 (2001), particularly pages 369-380 and 398-404).

A detectable measure of a CVD risk factor can be compared to the detectable measure of an appropriate control. An appropriate control can be the detectable measure of a CVD risk factor in the same mammal before treatment, or can be the detectable level of another mammal demonstrating similar CVD risk factors given a placebo.

Compositions described herein can be used in methods to increase in vivo and in vitro bioavailability (e.g., the concentration available for uptake in the stomach and intestine) of sterol compounds (e.g., phytosterol compounds) and/or fatty acid compounds (e.g., ω-3 fatty acid compounds). Compositions containing one or more phytosterol compounds and/or ω-3 fatty acid compounds in combination with a small carboxylic acid ester and/or a surfactant described herein can demonstrate improved bioavailability of phytosterol and/or ω-3 fatty acid compounds. See Example 19, below. Improved bioavailability of phytosterols can be evaluated relative to a control composition lacking a small carboxylic acid ester and/or surfactant, and can be determined by methods known to those of ordinary skill in the art, e.g., in an in vitro assay simulating the stomach and/or intestine environments.

The invention also provides a method for improving bioavailability of a sterol compound such as a phytosterol compound. The method includes incorporating a small carboxylic acid ester in any of the compositions described herein. For example, a composition exhibiting improved bioavailability of a phytosterol compound can include a phytosterol, an ω-3 fatty acid, and a small carboxylic acid ester. Another aspect of the invention provides a method for improving bioavailability of phytosterol compounds by incorporating a surfactant in any of the compositions described herein. For example, a composition exhibiting improved bioavailability of a phytosterol compound can include a phytosterol compound, an ω-3 fatty acid compound, and a surfactant. In other embodiments, both a small carboxylic acid ester and a surfactant can be included in a composition having a phytosterol compound and an ω-3 fatty acid compound.

In another aspect, the invention provides a method for improving bioavailability of ω-3 fatty acid compounds. The method includes incorporating a small carboxylic acid ester in any of the compositions described herein. For example, a composition exhibiting improved bioavailability of an ω-3 fatty acid compound can include a phytosterol compound, an ω-3 fatty acid compound, and a small carboxylic acid ester. Another aspect of the invention provides a method for improving bioavailability of an ω-3 fatty acid compound by incorporating a surfactant in any of the compositions described herein. For example, a composition exhibiting improved bioavailability of an ω-3 fatty acid compound can include a phytosterol compound, an ω-3 fatty acid compound, and a surfactant. In other embodiments, both a small carboxylic acid ester and a surfactant can be included in a composition having a phytosterol compound and an ω-3 fatty acid compound.

Assays for measuring bioavailability and bioabsorption in the stomach and intestine are known in the art (Wrobel et al., Biol. Trace Element Research (1999), p. 97; Motzok et al., J. Assoc. Anal. Chem. (1978), p. 887; Powell et al., Analyst (1998), p. 1721; Johnson, Biol. Trace Element Research (1989), p. 3; Hazel et al., British J. Nutr. (1987), p. 223; Miller et al., Amer. J. Clinical Nutr. (1981), p. 2248; Oomen et al., Environ. Sci. Tech (2002), p. 3326).

Methods of Administration

The compositions provided herein can be ingested (e.g., orally or intragastrically). The route of administration can depend on a variety of factors, such as the health of the patient and the therapeutic goals. One method of administration is orally in the form of capsules at daily-recommended dosages. Capsules are provided in sizes that are acceptable for the consumer to swallow. For example, capsules can be in the range from about 250 mg to 3 g in size, or any size therebetween (e.g., 275 mg, 300 mg, 350 mg, 400 mg, 450 mg, 475 mg, 490 mg, 500 mg, 550 mg, 575 mg, 600 mg, 650 mg, 675 mg, 700 mg, 750 mg, 800 mg, 900 mg, 1000 mg, 1250 mg, 1500 mg, 1750 mg, 1800 mg, 1900 mg, 2 g, 2.1 g, 2.2 g, 2.3 g, 2.4 g, 2.5 g, 2.6 g, 2.7 g, 2.8 g, or 2.9 g).

A composition provided herein can be provided in the form of a gel cap, soft gelatin capsule (e.g., soft gel capsule), or hard gelatin capsule. For example, a composition described herein can be encapsulated by a hard gel capsule. For oral administration, soft gel capsules can be prepared by conventional means with pharmaceutically acceptable excipients such as binding agents, fillers, lubricants, disintegrants, or wetting agents. Soft gel capsule manufacturing methods are described in, e.g., U.S. Pat. No. 6,333,047.

The compositions provided herein can be formulated as capsules that can demonstrate improved shelf life, homogeneity, and product stability. Phytosterols and fish oils were found to be not miscible. Composition can be designed to contain fillers such as beeswax, camauba wax, and other polymeric media to mask any separation of formulations, e.g., in soft gel capsules. The addition of such fillers can require the use of larger capsules or an increase in dose to obtain therapeutic (e.g., effective) levels of active ingredients.

In one aspect, the invention provides a method to stabilize (or to improve the stability of) a composition described herein that is lacking beeswax or carnauba wax by incorporating a small carboxylic acid ester in the composition. A composition incorporating a small carboxylic acid ester can be more stable than a composition lacking a small carboxylic acid ester in that (1) the composition is miscible and (2) the composition does not separate into separate phases in less than 24 hours, or less than 1 week, or less than 1 month. Typically, compositions containing small carboxylic acid esters do not require beeswax or carnauba wax to promote stability. See Example 21, below. A small carboxylic acid ester can be present in an amount of less than about 5% by weight of the composition, or less than about 2% by weight, or less than about 1% by weight. Particularly useful carboxylic acid esters include, without limitation, triethyl citrate, diethyl maleate, and diethyl L-tartrate.

Food grade surfactants can also stabilize the compositions described herein. A surfactant can be less than 10% by weight of the composition, or less than 5%, or less than 2%. Particularly useful surfactants include, without limitation, sorbitan derivatives, such as sorbitan oleate.

The invention will be further described in the following examples, which do not limit the scope of the invention described in the claims.

### EXAMPLES

### Example 1

A soft gel capsule containing:

| Ingredients | Amount, mg |
|---|---|
| Phytosterol Esters (>90%; | 567 |
| Cognis) | |
| 30% Fish oil (EPA:DHA 1.5:1 | 433 |
| (18%:12%)) | |
| Lecithin | 5 |
| CoQ-10 | 5 |
| Bromelain | 10 |

Serving recommendation is 3 capsules per day.

### Example 2

A soft gel capsule containing:

| Ingredients | Amount, mg |
|---|---|
| Phytosterol Esters (>94%, | 425 |
| Cognis) | |
| EPA | 88 |
| DHA | 58 |
| Polysorbate 20 | 10 |

Serving recommendation is 4 capsules per day.

### Example 3

A soft gel capsules containing:

| Ingredients | Amount, mg |
|---|---|
| Phytosterol Esters (>94%, | 567 |
| Cognis) | |
| EPA | 117 |
| DHA | 77 |

Serving recommendation is 3 capsules per day.

### Example 4

A soft gel capsule containing:

| Ingredients | Amount, mg |
|---|---|
| Phytosterol Esters (>94%, | 567 |
| Cognis) | |
| EPA | 117 |
| DHA | 77 |
| Acyl-lactylate | 10 |
| bromelain | 10 |

Serving recommendation is 3 capsules per day.

### Example 5

A hard shell gel capsule containing:

| Ingredients | Amount, mg |
|---|---|
| Phytosterols (>90%, Cognis) | 400 |
| Fish oil powder (EPA:DHA | 350 |
| 1.5:1) | |
| Polysorbate 20 | 5 |
| CoQ-10 | 5 |
| Bromelain | 10 |

Serving recommendation is 3 capsules per day.

### Example 6

A hard shell gel capsule containing:

| Ingredients | Amount, mg |
|---|---|
| Phytosterols (>90%) | 400 |
| Fish oil powder (EPA:DHA | 350 |
| 1.5:1) | |
| Lecithin | 5 |
| Tocotrienol | 7 |
| Bromelain | 8 |

Serving recommendation is 3-4 capsules per day.

### Example 7

A hard shell gel capsule containing:

| Ingredients | Amount, mg |
|---|---|
| Phytosterols (>90%) | 400 |
| Fish oil powder (EPA:DHA | 350 |
| 1.5:1) | |
| Lecithin | 5 |
| CoQ-10 | 5 |

Serving recommendation is 3 capsules per day.

### Example 8

A soft gel capsule containing:

| Ingredients | Amount, mg |
|---|---|
| Phytosterol Esters (>94% | 567 |
| phytostanol esters) | |
| EPA | 60 |
| DHA | 40 |
| Polysorbate 80 | 5 |
| Bromelain | 5 |

Serving recommendation is 3 capsules per day.

### Example 9

A soft gel capsule containing:

| Ingredients | Amount, mg |
|---|---|
| Phytostanol Esters (>92%) | 567 |
| EPA | 60 |
| DHA | 40 |
| Polysorbate 20 | 5 |
| Bromelain | 10 |

Serving recommendation is 3 capsules per day.

### Example 10

A soft gel capsule containing the following was made:

| Ingredients | Amount, mg |
|---|---|
| Phytosterol Esters (>90%) | 375 |
| Fish oil (30%; EPA:DHA | 275 |
| 18%:12%) | |
| Bromelain | 13 |
| Lecithin | 15 |
| Ubiquinone Q-10 | 5 |
| Triethyl citrate | 5 |
| Sorbitan oleate | 10 |

Serving recommendation is 4 capsules per day.

### Example 11

A soft gel capsule containing the following was made:

| Ingredients | Amount, mg |
|---|---|
| Phytosterol Esters (>90%) | 375 |
| Fish oil (30%; EPA:DHA | 275 |
| 18%:12%)) | |
| Bromelain | 13 |
| Lecithin | 15 |
| Triethyl citrate | 5 |
| Sorbitan oleate | 10 |

Serving recommendation is 4 capsules per day.

### Example 12

A soft gel capsule containing:

| Ingredients | Amount, mg |
|---|---|
| Phytosterol Esters (>90%) | 375 |
| Fish oil (30%, EPA:DHA | 275 |
| 18%:12%) | |
| Bromelain | 13 |
| Lecithin | 15 |
| Ubiquinone Q-10 | 5 |
| Triethyl citrate | 5 |
| Sorbitan oleate | 10 |
| Lemon oil | 6 |

Serving recommendation is 4 capsules per day.

### Example 13

A nutritional supplement containing:

SUPPLEMENT INGREDIENTS
Serving Size: 3-4 capsules

| Ingredient | Specs | Amount Per Serving | | % Daily Value* |
|---|---|---|---|---|
| Phytosterol esters | >94% | 1.7 | g | ** |
| Omega-3 Fatty Acid | | 300 | mg | ** |
| (triglyceride form) | | | | |
| EPA | | 180 | mg | ** |
| DHA | | 120 | mg | ** |
| Bromelain | | 50 | mg | ** |
| Lecithin | | 15 | mg | |
| CoQ-10 | | 10 | mg | ** |

*Percent Daily Value is based on a 2000 calorie diet (US RDA).
**Daily Value not established.

### Example 14

A nutritional supplement containing:

SUPPLEMENT INGREDIENTS
Serving Size: 3-4 Capsules

| | Specs | Amount Per Serving | | % Daily Value* |
|---|---|---|---|---|
| Phytosterol esters | >94% | 1.7 | g | ** |
| Omega-3 Fatty Acid | | 300 | mg | ** |
| (triglycerides form) | | | | |
| EPA | | 180 | mg | ** |
| DHA | | 120 | mg | ** |
| Bromelain | | 50 | mg | ** |
| Polysorbate-20 | | 10 | mg | |

*Percent Daily Value is based on a 2000 calorie diet (US RDI).
**Daily Value not established.

### Example 15

A nutritional supplement containing:

SUPPLEMENT INGREDIENTS
Serving Size: 3-4 capsules

| | Specs | Amount Per Serving | | % Daily Value* |
|---|---|---|---|---|
| Phytosterol esters | >94% | 1.7 | g | ** |
| Omega-3 Fatty Acid | | 300 | mg | ** |
| (triglycerides form) | | | | |
| EPA | | 180 | mg | ** |
| DHA | | 120 | mg | ** |
| Lecithin | | 25 | mg | |

*Percent Daily Value is based on a 2000 calorie diet (US RDA).
**Daily Value not established.

### Example 16

A nutritional supplement containing the following was made:

SUPPLEMENT INGREDIENTS
Serving Size: 3-4 capsules

| | Specs | Amount Per Serving | | % Daily Value* |
|---|---|---|---|---|
| Phytosterol esters | >94% | 1.7 | g | ** |
| Omega-3 Fatty Acid | | 300 | mg | ** |
| (triglycerides form) | | | | |
| EPA | | 180 | mg | ** |
| DHA | | 120 | mg | ** |
| Bromelain | | 50 | mg | ** |
| Sorbitan oleate | | 40 | mg | |
| Triethyl Citrate | | 20 | mg | |
| CoQ-10 | | 5 | mg | ** |

*Percent Daily Value is based on a 2000 calorie diet (US RDA).
**Daily Value not established.

### Example 17

A nutritional supplement containing:

SUPPLEMENT INGREDIENTS
Serving Size: 3-4 capsules

| | | |
|---|---|---|
| Specs | Amount Per Serving | % Daily Value* |

| | | | | |
|---|---|---|---|---|
| Phytosterol esters | >94% | 1.7 | g | ** |
| Omega-3 Fatty Acid | | 300 | mg | ** |
| (triglycerides form) | | | | |
| EPA | | 180 | mg | ** |
| DHA | | 120 | mg | ** |

*Percent Daily Value is based on a 2000 calorie diet (US RDA).
**Daily Value not established.

### Example 18

### Soft Gel Cap Preparation

The following procedure was used to encapsulate soft gel caps. The capsule ingredients were weighed and compounded by shearing in a vacuum mixture until homogeneous. The mixture was cooled to 31-34° C. A gelatin mixture (containing gelatin, colorant, glycerin, and water) was then heated, sheared and kept under vacuum in the vacuum mixer until any foam subsided. The gelatin mixture was held at >34° C. A Kamata or Bo Cheng encapsulator was used, with die roll size 18 oval (or oblong), segment size 14 oval (or oblong). The mix and gelatin mix were charged into the batch. The capsules were sampled and inspected. Capsules were dried on trays to a final moisture content of 5-10%.

### Example 19

### In Vitro Absorption Measurement

Omega-3 fatty acid and phytosterol bioavailability (e.g., amount available for absorption through the stomach and/or intestine) were examined for various compositions described herein using an in vitro method simulating the stomach and intestine environments. Four compositions were examined: one containing phytosterol esters (PSE) only; one containing fish oil only; one containing PSE and fish oil; and Example 10, above.

In a 150 ml beaker, a 100 g stock pepsin solution was prepared and the pH adjusted to 1.8. In a 400 ml beaker, a 200 g 0.1 M solution of HCl (pH 1.8) was prepared. A constant temperature water bath was heated to 37° C. The 0.1M HCl solution was placed into the water bath so that the water level in the bath was about one inch below the top of the beaker. 25 ml of the pepsin solution and 30 g of a composition were added to a dialysis tube. An initial 10 ml aliquot (To) of the 0.1M HCl solution in the water bath was taken. The dialysis tube was placed into the 0.1 M HCl solution and allowed to react for 1 hour with occasional mixing. After one hour, another 10 ml aliquot (T₁) of the 0.1M HCl solution was taken. The HCl solution was then neutralized with sodium hydroxide (NaOH) and the pH was adjusted to approximately 6.0. 0.5 g of pancreatin was added to the solution in the dialysis bag and mixed thoroughly. Aliquots of the pancreatin mixture were taken at 2 hours (T₂) and 4 hours (T₃). Gas chromatography was used to measure the amount of phytosterol (PS) and phytosterol esters (PSE) in each aliquot. See Table 1 below.

TABLE 1
GC Analysis of Aliquots at Various Time Points

| Sample | T₀ | T₁ | T₂ | T₃ |
|---|---|---|---|---|
| Phytosterol | PSE: 0 mg/g | PSE: 0 mg/g | PSE: 0 mg/g | PSE: 0 mg/g |
| ester only | PS: 0 mg/g | PS: 0 mg/g | PS: 0 mg/g | PS: 0 mg/g |
| Fish oil only | PSE: 0 mg/g | PSE: 0 mg/g | PSE: 0 mg/g | PSE: 0 mg/g |
| | PS: 0 mg/g | PS: 0 mg/g | PS: 0 mg/g | PS: 0 mg/g |
| Phytosterol | PSE: 0 mg/g | PSE: 0 mg/g | PSE: 0 mg/g | PSE: 0 mg/g |
| ester and | PS: 0 mg/g | PS: 0 mg/g | PS: 0 mg/g | PS: 0 mg/g |
| fish oil only | | | | |
| Example 10 | PSE: 0 mg/g | PSE: 0 mg/g | PSE: 0 mg/g | PSE: 0.11 mg/g |
| | PS: 0 mg/g | PS: 0 mg/g | PS: 0 mg/g | PS: 0.25 mg/g |

In Table 1, T₀ represents the unreacted sample; T₁ represents the sample after acid digestion in the stomach; T₂ represents the sample after 2 hours in a small intestine environment, and T₃ represents the sample after 4 hours in a small intestine environment.

These results demonstrate that fish oil alone did not increase the bioavailability of phytosterol or phytosterol esters in this absorption test. Even in harsh gastric conditions, no detectable phytoserol or phytosterol esters were seen at the four time points. The composition of Example 10, however, containing triethyl citrate, a small carboxylic acid ester, and sorbitan oleate, a surfactant, demonstrated surprisingly improved bioavailability of phytosterol and phytosterol esters.

### Example 20

### Gas Chromatography Method

This GC-method quantitatively determined the amount of free sterols, the sum of free sterols plus sterol esters, and the free fatty acids in the aliquots taken in Example 19.

The Grunau Analytical Method IA 1117-4117 was used, along with a GLC HP 5890 Series II. The sample was dissolved in pyridine and silylated with MSTFA and separated on GC by split injection on a DB 5 HT capillary column using a temperature program to 370° C. with FID detection. Quantitative evaluation of free sterols and fatty acids was done using an internal standard on the basis of multiple point calibration. The amount of sterol esters was represented by area percentage of all esters in the chromatogram.

### Example 21

### Evaluation of Composition Stability

Varying amounts of PSE and fish oil were mixed in a 50 ml beaker. The mixture was warmed to 35-40° C. A variety of test reagents, including surfactants and small carboxylic acid esters, were then examined for their ability to stabilize the mixture at room temperature (RT), 40° C., and 50° C. The resulting mixtures were classified as follows: insoluble (separated immediately); unstable (separated quickly, usually less than 1-2 hr); stable (did not separate for 24 hours); very stable (did not separate for over 1 week). See Table 2, below.

TABLE 2
Stability Data

| | Fish | | Test Reagent | |
|---|---|---|---|---|
| Phytosterol | Oil | Test Reagent | amount | Results |
| 4 g | 2 g | None | None | insoluble |
| 2 g | 2 g | None | None | insoluble |
| 2 g | 4 g | None | None | insoluble |
| 4 g | 2 g | Polysorbate 20 | 0.10 g | unstable |
| 4 g | 2 g | Polysorbate 20 | 0.20 g | unstable |
| 2 g | 2 g | Polysorbate 60 | 0.10 g | unstable |
| 4 g | 2 g | Polysorbate 60 | 0.20 g | unstable |
| 4 g | 2 g | Polysorbate 60 | 0.30 g | unstable |
| 4 g | 2 g | Polysorbate 60 | 0.40 g | unstable |
| 2 g | 2 g | Polysorbate 80 | 0.10 g | unstable |
| 4 g | 2 g | Polysorbate 80 | 0.20 g | unstable |
| 4 g | 2 g | Glycerin | 0.10 g | insoluble |
| 2 g | 2 g | Triethyl Citrate | 0.30 g | unstable |
| 4 g | 2 g | Triethyl Citrate | 0.10 g | stable |
| 4 g | 2 g | Triethyl Citrate | 0.20 g | stable |
| 4 g | 2 g | Triethyl Citrate | 0.30 g | stable |
| 4 g | 2 g | Triethyl Citrate | 0.40 g | stable |
| 4 g | 2 g | Triethyl Citrate | 0.05 g | very stable |
| | | Sorbitan Oleate | 0.05 g | |
| 4 g | 2 g | Triethyl Citrate | 0.10 g | very stable |
| | | Sorbitan Oleate | 0.10 g | |
| 4 g | 2 g | Triethyl Citrate | 0.20 g | very stable |
| | | Sorbitan Oleate | 0.20 g | |
| 4 g | 2 g | Triethyl Citrate | 0.05 g | very stable |
| | | Sorbitan Laurate | 0.05 g | |
| 4 g | 2 g | Triethyl Citrate | 0.10 g | very stable |
| | | Sorbitan Laurate | 0.10 g | |
| 4 g | 2 g | Triethyl Citrate | 0.20 g | very stable |
| | | Sorbitan Laurate | 0.20 g | |
| 4 g | 2 g | Sorbitan Oleate | 0.10 g | unstable |
| 4 g | 2 g | Sorbitan Oleate | 0.20 g | unstable |
| 4 g | 2 g | Sorbitan Oleate | 0.30 g | unstable |
| 4 g | 2 g | Sorbitan Oleate | 0.40 g | unstable |
| 4 g | 2 g | Decyl Glucoside, | 0.10 g | unstable |
| | | Sodium Lauroyl | | |
| | | Lactylate | | |
| 4 g | 2 g | Sodium Isostearyl | 0.10 g | unstable |
| | | Lactylate | | |
| 4 g | 2 g | Cocamidopropyl | 0.20 g | unstable |
| | | Betaine, Sodium | | |
| | | Lauroyl Lactylate | | |
| 4 g | 2 g | Sodium Isostearoyl | 0.20 g | unstable |
| | | Lactylate, Propylene | | |
| | | Glycol | | |
| 4 g | 2 g | Sodium Caproyl | 0.20 g | unstable |
| | | Lactylate | | |
| 4 g | 2 g | Sodium C14-16 Olefin | 0.20 g | unstable |
| | | Sulfate, Cocamidopropyl | | |
| | | Betaine, Sodium | | |
| | | Lauroyl Lactylate, PEG- | | |
| | | 150 Distearate | | |
| 4 g | 2 g | Citric Acid | 0.10 g | insoluble |
| 4 g | 2 g | Citric Acid | 0.20 g | insoluble |
| 4 g | 2 g | Citric Acid | 0.30 g | insoluble |
| 4 g | 2 g | Diethyl Maleate | 0.10 g | stable |
| 4 g | 2 g | Diethyl Maleate | 0.20 g | stable |
| 4 g | 2 g | Diethyl Maleate | 0.30 g | insoluble |
| 4 g | 2 g | Diethyl L-tartrate | 0.10 g | stable |
| 4 g | 2 g | Diethyl L-tartrate | 0.20 g | stable |
| 4 g | 2 g | Diethyl L-tartrate | 0.30 g | stable |
| 4 g | 2 g | Triethyl Citrate | 0.30 g | stable |
| 4 g | 2 g | Triethyl Citrate, | 0.30 g | very stable |
| | | Sorbitan Oleate | 0.30 g | |
| 4 g | 2 g | Sorbitan Oleate | 0.30 g | unstable |

These results demonstrate that the addition of small amounts (e.g., about 5% or less of the composition, by weight) of a small carboxylic acid ester such as diethyl tartrate or triethyl citrate can significantly stabilize the compositions described herein. The addition of small amounts (e.g., about 5% or less of the composition, by weight) of a food grade surfactant such as sorbitan oleate or sorbitan laurate can further improve stability. The addition of these ingredients in small quantities, typically less than 5% of the combined amount of phytosterol ester and fish oil mixture, can result in a stable and homogenous formulation.

### Example 22

### Case Studies

A 43 year old Asian man demonstrating high cholesterol (>250 mg/dL) for over a decade was administered a supplement as described in Example 10 (4 soft gel capsules/day for 2 months). Results are shown below in Table 3.

TABLE 3
Blood Lipid Panel

| Blood | | | | |
|---|---|---|---|---|
| Detectable | Before | After | Percentage | Recommended |
| Measure | Supplement | Supplement | Change | Level* |
| Total | 252 mg/dL | 211 mg/dL | −16.27% | |
| Cholesterol | | | | |
| LDL | 180 mg/dL | 139 mg/dL | −22.78% | <130 |
| HDL | 58 mg/dL | 51 mg/dL | −12.07 | >40 mg/dL |
| Triglycerides | 70 mg/dL | 101 mg/dL | +44.3 | <150 mg/dL |
| C-Reactive | 0.17 mg/dL | 0.1 mg/dL | −41.18% | |
| Protein (CRP) | | | | |

*JAMA vol. 285 p. 2486 (2001).

These results demonstrate that significant reductions were seen for the subject's total cholesterol (greater than 16% reduction) and LDL cholesterol (greater than 22% reduction) levels.

A 44 year old Caucasian male was administered a supplement as described in Example 10 (4 soft gel capsules/day for 3 weeks). Results are shown below in Table 4.

TABLE 4
Blood Lipid Panel

| Blood Detectable | | | Percentage |
|---|---|---|---|
| Measure | Before Supplement | After Supplement | Change |
| Total Cholesterol | 229 mg/dL | 219 mg/dL | −4.4% |
| LDL | 159.9 mg/dL | 135.6 mg/dL | −15.2% |
| HDL | 43.7 mg/dL | 40.0 mg/dL | −8.46% |

These results demonstrate that a significant reduction (greater than 15%) was seen for the subject's LDL cholesterol level.

### Example 23

### Design of a Human Clinical Trial

A pilot study was designed to determine the efficacy of various test compositions after 4 and 8 weeks use on a panel of healthy volunteers between the ages of 18 and 55 years. The study is conducted in accordance with the intent and purpose of Good Clinical Practice regulations described in Title 21 of the U.S. Code of Federal Regulations (CFR).

The test compositions were as follows:

| | |
|---|---|
| Percentage | Mg/capsule |

Phytosterol ester formula (Placebo A) - dosage 4 capsules/day

| | | |
|---|---|---|
| Description | | |
| Refined coconut oil (Miglyol | 5.33% | 37.5 |
| 812) | | |
| Phytosterol Esters, (VegaPure, | 54.69% | 385.0 |
| Cognis, Lot GR21413161) | | |
| Annatto Oil Concentrate | 0.07% | 0.5 |
| Fish Oil 30% Omega 3 Fatty | 39.06% | 275.0 |
| Acids (DeNofa, Lot D20118- | | |
| 05) | | |
| Lemon Flavor (Natural, Oil | 0.85% | 6.0 |
| Soluble) | | |
| Shell Description | | |
| Gelatin, Lime bone 150 | 43.23% | |
| Bloom | | |
| Glycerin 99.5% USP (RM | 19.92% | |
| 6003) | | |
| Purified Water USP | 36.45% | |
| Carmine Powder CC- | 0.40% | |
| 10WWS-P | | |

Phytosterol ester formula (Placebo B) - dosage 4 capsules/day

| | | |
|---|---|---|
| Description | | |
| Refined Coconut Oil (Miglyol | 45.81% | 322.5 |
| 812) | | |
| Phytosterol Esters, (VegaPure, | 53.27% | 375.0 |
| Cognis, Lot GR21413161) | | |
| Annatto Oil Concentrate | 0.07% | 5.0 |
| Lemon Flavor (Natural) Oil | 0.85% | 6.0 |
| Soluble | | |
| Shell Description | | |
| Gelatin, Lime Bone 150 Bloom | 43.23% | |
| Glycerin 99.5% USP (RM 6003) | 19.92% | |
| Purified Water USP | 36.45% | |
| Carmine Powder CC-10WWS-P | 0.40% | |

Fish Oil Formula (Placebo C) - dosage 4 capsules/day

| | | |
|---|---|---|
| Description | | |
| Refined coconut oil | 50.92% | 358.5 |
| (Miglyol 812) | | |
| Annatto Oil Concentrate | 0.07% | 0.5 |
| Beeswax, Yellow | 9.09% | 64.0 |
| Fish Oil 30% Omega 3 | 39.06% | 275.0 |
| Fatty Acids (Denofa, Lot | | |
| D20118-05) | | |
| Lemon Flavor (Natural) | 0.85% | 6.0 |
| Oil Soluble | | |
| Shell Description | | |
| Gelatin, Lime Bone 150 | 43.23% | |
| Bloom | | |
| Glycerin 99.5% USP (RM | 19.92% | |
| 6003) | | |
| Purified Water USP | 36.45% | |
| Carmine Powder CC- | 0.40% | |
| 10WWS-P | | |

Phytosterol Ester/Fish Oil Formula - dosage 4 capsules/day

| | | |
|---|---|---|
| Description | | |
| Phytosterol Esters, | 53.27% | 375.0 |
| (VegaPure, Cognis, Lot | | |
| GR 21413161) | | |
| Fish Oil 30% Omega 3 | 39.06% | 275.0 |
| Fatty Acids (Denofa, Lot | | |
| D20118-05) | | |
| Bromelain Powder 600 | 1.85% | 13.0 |
| GDU/gm (900 MCU) | | |
| Lecithin Lecicap NV | 2.13% | 15.0 |
| Coenzyme Q10 100% | 0.71% | 5.0 |
| Pure Crystalline | | |
| Triethyl Citrate | 0.71% | 5.0 |
| Sorbitan Oleate | 1.42% | 10.0 |
| Lemon Flavor (Natural) | 0.85% | 6.0 |
| Shell Description | | |
| Gelatin, Lime Bone 150 | 43.23% | |
| Bloom | | |
| Glycerin 99.5% USP (RM | 19.92% | |
| 6003) | | |
| Purified Water USP | 36.45% | |
| Carmine Powder CC- | 0.40% | |
| 10WWS-P | | |

All test compositions were manufactured using the same lots of raw materials. Random samples were analyzed for phytosterol ester, DHA, and EPA concentrations before distributing to subjects. The test compositions were packaged and labeled (coded) in a manner consistent with the study design. Test compositions were distributed only to subjects entered into the study, under the supervision of the Investigator, in accordance with the conditions specified below.

Approximately 240 subjects initially volunteered for the study. Height, weight, and blood pressure measurements were taken, and the subjects were given questionnaires regarding their life style, disease history, and dietary habits. Informed consent was obtained from each subject and documented in writing before participation in the study. A copy of the informed consent form was provided to each subject.

To be enrolled in the trial, volunteer subjects had to:
be between the ages of 18 and 55 years and in general good health;
acknowledge that they had a borderline or slightly high blood total cholesterol level;
be free of any cardiovascular disease and other chronic disease, including a known history of allergies or other medical conditions, which, in the opinion of the Investigator, could have interfered with the conduct of the study or the interpretation of results, or could have increased the risk of adverse reactions;
understand and provide written informed consent;
anticipate ability to complete the course of the study and to comply with instructions; and
be willing to donate blood samples for blood chemistry work.

The following subjects were excluded from the study: females who were pregnant or nursing or who anticipated becoming pregnant during the experimental period; regular smokers; subjects who suffered from gastrointestinal complaints; subjects taking prescribed medications (i.e. statins) that might interfere with the test results; subjects who were currently under treatment for asthma or diabetes; subjects exceeding intensive exercise for time periods greater than 10 hr/wk; subjects using phytosterol spreads, including “Benecol” or “Take Control;” subjects who suffered from high blood pressure or had high blood cholesterol (>250 mg/dL); subjects who were on a diet or special diet for medical reasons; subjects taking aspirin or other NSAIDs; subjects taking ProvexCV™; and regular wine drinkers.

Based on a review of the questionnaires and consent agreements, blood samples were drawn from 139 volunteer subjects. After the blood samples were analyzed, about 40 subjects remained as suitable for enrollment in the trial.

For the trial, the subjects were divided into four groups, with each group given one of the four test compositions (dosage=1 capsule 4×/day) randomly. Both the subjects and Investigator were double-blinded. Subjects were instructed to refrain from using any prescription drugs, special diets, or other phytosterol or fish oils products. Each subject was given the coded and randomized test composition, a daily diary, and an instruction form.

Subjects were instructed to report to a testing facility after 4 and 8 weeks of product use. Blood was drawn and weight, height, and blood pressure measurements taken. The technicians submitted the code and blood samples to qualified medical laboratories for analyses of total blood cholesterol, HDL, LDL, triglycerides, and C-reactive proteins. At the final visit, subjects were required to respond to a brief questionnaire.

Upon completion, the test compositions and subjects are decoded and reviewed. All compositions are decoded, reviewed, and quality-proofed by technicians to ensure accuracy. Technicians generate a log and spread sheet containing subject names, product code, weight, height, BMI, and blood test results. The Investigator decoded the results after 4 weeks (see Example 24 below) and would also decode the results after 8 weeks.

An average of each of the total blood cholesterol, LDL, HDL, triglycerides, and C-reactive protein levels is determined, and the arithmetic mean change in lipid levels, standard deviations from the mean, and probabilities from the paired-difference t-test and Wilcoxon signed-rank tests are determined for the change in lipids and C-reactive proteins between 0, 4, and 8 weeks. Statistical significance is denoted at the p<0.05 level.

### Example 24

### Results of Clinical Trial of Example 24 After 4 Weeks

Data from the clinical trial of Example 24 were evaluated after 4 weeks of use. There was no significant baseline difference among the groups with regard to age, body-mass index (BMI), and gender (Table 5). Ninety four percent of the subjects completed the study. Three subjects were eliminated when they increased their exercise and changed their diet. There was >95% compliance with the study regimen in all the groups.

TABLE 5
Demographic Baseline Data

| | Formula A | Formula B | Formula C | Formula D |
|---|---|---|---|---|
| n | 11 | 10 | 11 | 10 |
| Average Age | 39.2 | 45.2 | 42.7 | 36.4 |
| Gender (M/F) | 6/5 | 6/4 | 6/5 | 6/4 |
| BMI | 31 | 29 | 28 | 29 |
| Average TC | 204 | 216 | 209 | 232 |
| (mg/dL) | | | | |
| Average HDL | 39 | 42 | 42 | 44 |
| (mg/dL) | | | | |
| Average LDL | 149 | 160 | 150 | 169 |
| (mg/dL) | | | | |
| Average TG | 146 | 123 | 140 | 160 |
| (mg/dL) | | | | |
| Average | 0.38 | 0.27 | 0.28 | 0.31 |
| CRP(mg/dL) | | | | |

Averages of the total serum cholesterol, LDL, HDL, triglycerides, and C-reactive protein levels were determined. The arithmetic mean change, standard deviation from the mean, and probabilities from the paired-difference t-test were determined for the change in lipid levels and C-reactive protein.

Serum Total Cholesterol Levels

There was a statistical difference in serum total cholesterol changes after 4 weeks. Subjects administered Formula D showed an average reduction of 9 mg/dL, and 80% of subjects administered Formula D saw a reduction in total cholesterol. Certain subjects administered Formula D saw a reduction of >40 mg/dL in total cholesterol. Subjects administered Formula A showed an average reduction of 3 mg/dL, and 70% of Formula A subjects saw a reduction in total cholesterol. Subjects administered Formula B did not exhibit a reduced average total cholesterol level. Subjects administered Formula C exhibited an increase in total cholesterol levels.

HDL, LDL, and Triglyceride Levels

There were no statistical differences in HDL levels after 4 weeks for any of the subjects. LDL levels did exhibit statistical differences after 4 weeks. Subjects administered Formula D demonstrated an average reduction of 10 mg/dL in LDL levels, with certain subjects demonstrating a reduction of up to 40 mg/dL. Subjects administered Formula A demonstrated an average reduction of 6 mg/dL in LDL levels. There was a trend that subjects administered Formulas D, A, and C exhibited reduced levels of triglycerides relative to those administered Formula B, but the results were not statistically significant.

C-Reactive Protein Levels

Subjects administered Formulas D and A demonstrated reduced CRP levels, although the changes were not statistically significant.

Conclusions

The data after 4 weeks demonstrate that formulas containing phytosterol and ω-3 fatty acids had a positive impact on risk markers associated with CVD. Patients administered such formulas demonstrated statistically significant reductions in total serum cholesterol and HDL levels, and reductions (although not necessarily statistically significant) in triglyceride and C-reactive protein levels.

OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A supplement comprising a composition comprising one or more sterol compounds and one or more fatty acid compounds, wherein a ratio of the total amount of sterol compounds to the total amount of fatty acid compounds is ≦about 5:1.

2. The supplement of claim 1, wherein said one or more sterol compounds are selected from the group consisting of phytosterols, phytosterol esters, phytostanols, and phytostanol esters, or combinations thereof, and wherein said one or more fatty acid compounds are selected from ω-3 fatty acid compounds.

3. The supplement of claim 2, wherein said composition is a homogeneous mixture.

4. The supplement of claim 1, wherein said supplement is in the form of a hard-shell capsule or a soft-gel capsule.

5. The supplement of claim 1, wherein said supplement includes a label recommending a serving size of at least 1 g of the one or more sterol compounds and/or at least 300 mg of the one or more fatty acid compounds per day.

6. The supplement of claim 2, wherein said one or more sterol compounds are phytosterol esters that represent from about 70% to about 85% by weight of said supplement.

7. The supplement of claim 2, wherein said one or more fatty acid compounds are ω-3 fatty acid compounds that represent from about 10% to about 55% by weight of said supplement.

8. The supplement of claim 7, wherein said ω-3 fatty acid compounds represent from about 10% to about 30% by weight of said supplement.

9. The supplement of claim 3, wherein said supplement comprises a small carboxylic acid ester.

10. The supplement of claim 2, wherein said ω-3 fatty acid compounds comprise ALA.

11. The supplement of claim 10, wherein said ALA is provided in a flax seed oil mixture.

12. The supplement of claim 5, wherein said wherein said supplement includes a label recommending a serving size of at least 1.5 g of the one or more sterol compounds per day, wherein said one or more sterol compounds comprise phytosterol ester compounds, and at least 300 mg of said one or more fatty acid compounds per day, wherein said at least 300 mg of said one or more fatty acid compounds comprises about 180 mg DHA and about 120 mg EPA.
